(19) United States
(12) Patent Application Publication
(10) Pub. No.: US 2025/0339333 A1
(43) Pub. Date: Nov. 6, 2025

(54) HANDHELD SKIN REJUVENATING DEVICE

(71) Applicant: Nastran Bolbolan, Apple Valley, CA (US)

(72) Inventor: Nastran Bolbolan, Apple Valley, CA (US)

(21) Appl. No.: 18/653,990

(22) Filed: May 3, 2024

(51) Int. Cl. A61H 9/00; A61N 1/32; A61N 5/06

(52) U.S. Cl. CPC A61H 9/0057; A61H 9/0007; A61N 1/328; A61N 5/0616; A61H 2201/0107; A61H 2201/0153; A61H 2201/0157; A61H 2201/10; A61H 2201/1207; A61H 2201/14; A61H 2201/1664; A61H 2201/1671; A61H 2201/1695; A61H 2201/5005; A61H 2205/022; A61H 2209/00; A61N 2005/0652; A61N 2005/0663

(57) ABSTRACT

The present invention relates hand-held skin rejuvenating devices with micro-current low-frequency pulse generators arranged in the tips of rejuvenation fingers. The present rejuvenation device has the advantage of a two-dimensional massaging feature, that uniformly rejuvenates skin to attain maximum rejuvenation. Additionally. the utility model provides a rejuvenation appearance, and the transmission of the rejuvenation appearance is vertical and horizontal.

The present complete specification is claiming priority from the provisional patent application U.S. Pat. No. 63,548,188 and EFSID 48820025 filed on 11-NOV-2023 by applicant NASTRAN BOLBOLAN.

### FIELD OF THEINVENTION

The present invention relates generally to the technical field of hand-held skin rejuvenation devices with micro-current low-frequency pulse generators with push and pull mechanisms.

### BACKGROUND

The skin rejuvenation or rejuvenation devices currently available in the market have the disadvantage of rotating uni-dimensionally, leading to nonuniform rejuvenation skin, thus not attaining desirable results. The present invention has the advantage of a two-dimensional massaging feature, that uniformly massages the skin attaining, maximum rejuvenation.

The utility model discloses, aiming to solve one of the technical problems, of the prior art. Therefore, the utility model provides a uniform rejuvenation, with the transmission of rotating appearance being vertical and horizontal movement simultaneously.

### SUMMARY OF THE INVENTION

The present invention relates generally to the technical field of hand-held skin rejuvenation or rejuvenation appliances in combination with micro-current low-frequency pulse generators with push and pull mechanisms.

One of the embodiments of the present invention relates to a skin rejuvenation utility device comprising the integration of three powerful technologies such as:
i. rejuvenation fingers with circular holes arranged similarly to a sieve,
ii. tiny protrusions moving vertically through circular holes offingers,
iii. micro-current, and;
iv. LED light (red, blue, and purple variations).

In one of the embodiments, the present invention relates to a hand-held massaging device including:
i. rejuvenation fingers, with circular holes arranged similarly, in a sieve or mesh, thus fingers arranged on the shell,
ii. driving motor fixed in the casing,
iii. LED light (red, blue, and purple variations) and;
iv. independent tiny protrusions with an independent vertical push and pull mechanism at regulated intervals.

One of the embodiments of the present invention relates to a rejuvenation device with fingers fixed to the shell with circular holes for facilitating the movement of silicon protrusions. Further, the utility model provides a uniform rejuvenation, a combination of a horizontal rotating appearance of fingers and vertical push and pull movement of silicon protrusions simultaneously. Additionally, the vertical push and pull mechanisms create a minimalistic suction effect on the skin, thus activating the localized blood flow in the localized area. Hence, the increased blood flow promotes and activates the skin rejuvenation.

The technical scheme adopted by the utility model is as follows: the rejuvenation device comprises an outer shell, a rotary driving mechanism, and a rejuvenation mechanism, wherein the rotary driving mechanism is arranged in the outer shell, the rejuvenation mechanism comprises three rejuvenation fingers with circular holes arranged uniformly for facilitating protrusions. The massager thus, creates a combined horizontal rotation with a vertical pulse of suction at regular intervals simultaneously.

In one of the embodiments of the present invention, rejuvenation fingers are attached to the shell with circular holes arranged as in a sieve, thus facilitating the vertical movement of silicon structures facilitating push and pull suctional movement at regulated time intervals. Additionally, three adjustable speed settings are accompanied by silicon structures delicately designed. They generate pull and push touch providing user a gentle lifting effect. All features that come with the device work together to tighten, decollate, and rejuvenate one's face and neck, enhancing the overall skin texture.

According to some embodiments of the present invention, the rejuvenation fingers and the rotation shaft can be electrically conductive, and an electrotherapy driving module electrically connected to the rotation shaft is provided in the housing.

One of the embodiments further to electrically conductive fingers with tiny protrusions, conduct microcurrent during rotation. Additionally, each finger has a removable sieved cap. Upon removing the cap, the silicon protrusions fixed to each finger can be cleaned and the cap can be replaced with a screw rotational lock to resume function.

### BRIEF DESCRIPTION OF THE DRAWINGS

FIG. 1A is a perspective view of the rejuvenation device.

FIG. 1B is a top view of the lid of the rejuvenation device.

FIG. 1C is a rear view of the lid of the rejuvenation device.

FIG. 2 A is a bottom plan view of the rejuvenation device.

FIG. 2 B is an exploded view of the hand-held rejuvenation device.

FIG. 2 C is the magnified side view of one of the three rejuvenation fingers.

FIG. 2 D is the magnified side view of the three fingers with silicon protrusions out of the sieved holes of each figure.

FIG. 3 A is an isometric diagonal left-side view of the push mechanism assembly fixed inside the rejuvenation housing.

FIG. 3 B is an isometric diagonal right-side view of the push mechanism assembly fixed inside the rejuvenation housing.

FIG. 3 C is a trimetric projection of the push mechanism assembly fixed inside the rejuvenation housing.

FIG. 4 is related to the circuit diagram concerning the working of the skin rejuvenation.

### DETAILED DESCRIPTION

Embodiments are provided to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, apparatus structures, and techniques are not described in detail.

The terminology used, in the present disclosure, is only to explain a particular embodiment and such terminology shall not be considered to limit the scope of the present disclosure. As used in the present disclosure, the forms “a,” “an,” and “the” may be intended to include the plural forms as well, unless the context suggests otherwise. The terms “comprises,” “comprising,” “including,” and “having,” are open-ended transitional phrases and therefore specify the presence of stated features, integers, steps, operations, elements, modules, units, and components, but do not forbid the presence or addition of one or more other features, integers, steps, operations, elements, components, and groups thereof.

The following are the definitions of the parts described supporting the invention aspects:
i. Rejuvenation device: A handheld rejuvenation needs to be held by the user and run massaging head over various body areas.
ii. Skin rejuvenation device: Skin rejuvenation usually refers to the treatment of the skin of the face, many are also appropriate for the treatment of skin on the body. Some treatments are topical, and administered to the upper layer of the skin of the affected area.
iii. Vertical rotation: It is a type of motion in which the distance of the body remains constant from a fixed plane.
iv. Push and pull or suction: The silicon protrusions, on each finger create a push-pull movement and create suction for a minimal period activating localized blood flow to the skin.
v. Microcurrent: Microcurrent is a non-invasive and safe electrotherapy applied through a series of sub-sensory electrical currents (less than 1 mA), which are of a similar magnitude to the currents generated endogenously by the human body.
vi. LED lights: A light-emitting diode (LED) is a semiconductor device that emits light when an electric current flows through it.
vii. DC motor: A DC motor is an electrical machine that converts electrical energy into mechanical energy. In a DC motor, the input electrical energy is the direct current which is transformed into the mechanical rotation.
viii. Resistor: A resistor is a passive two-terminal electrical component that implements electrical resistance as a circuit element. In electronic circuits, resistors are used to reduce current flow, adjust signal levels, divide voltages, bias active elements, and terminate transmission lines, among other uses.

### DETAILED DESCRIPTION OF FIGURES

The present invention will be further described concerning the following examples for facilitating the understanding of those skilled in the art, and the description of the embodiments is not intended to limit the present invention. One of the embodiments of the present invention relates to a hand-held facial massaging device with a detailed explanation of functional parts, with a list of figures below:

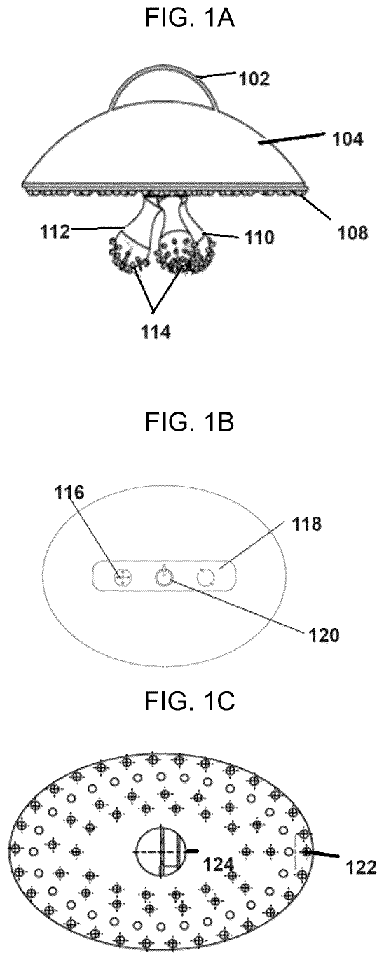

FIG. 1A is a perspective view of the rejuvenation device. FIG. 1, is elaborately explained to comprise 102 representing the handle, 104 representing the housing, and the soft rejuvenation fingers, comprised of the gearbox and the DC motor facilitating the rotational mechanism and 114 being the plural rejuvenation or massage fingers three in number respectively. The plurality of the fingers can be increased based on the enhancements of the product. The housing internally is provided with a control panel and micro-current low-frequency pulse-generating device. When a rejuvenation works at least three soft conductive fingers are electrically conducted and the microcurrent is passed through with three configurable rotational speeds. Further, 108 is the LED lights arranged on the lid of the rejuvenation device, 112 represents the neck of a finger and 110 is the attachment of the sieved cap of a finger to the neck of a finger.

FIG. 1B is a top view of the lid of the rejuvenation device. FIG. 1B is elaborately explained to comprise ‘116’ representing MODE functionality, wherein the term MODE is further defined as a configuration of applications or usage of skin/facial rejuvenation comprising of:
a. only horizontal rotation
b. combination of vertical and horizontal rotation,
c. only vertical rotation,
d. only LED usage, and;
e. a LED usage (selected color) combination, with any configurable rotational speeds. Further, ‘118’ is the rotation per minute controller, and ‘120’ represents the POWER switch for ON and OFF respectively.

FIG. 1C is a rear view of the lid of the rejuvenation device. ‘124’ represents LEDs arranged along the rim of the lid and ‘124’ as the central hole where the motor assembly from the outside and fingers from the inside are fixed.

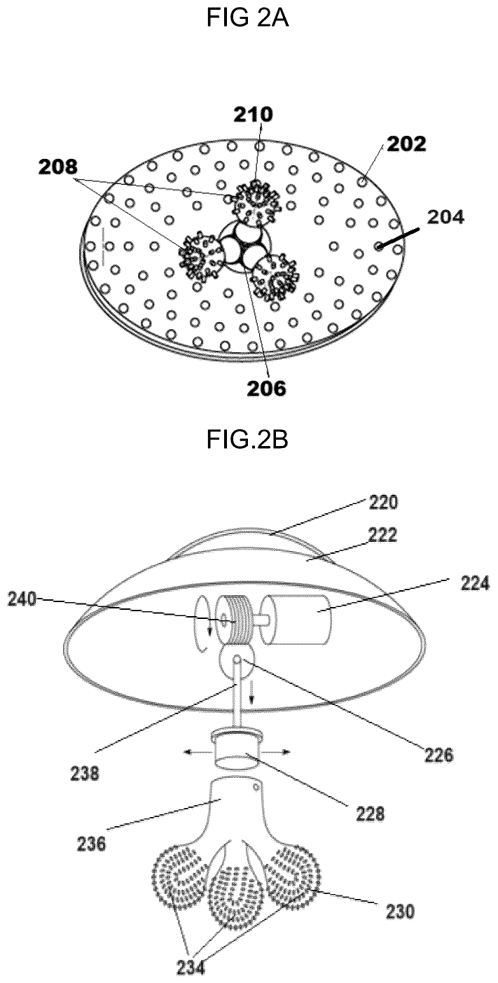

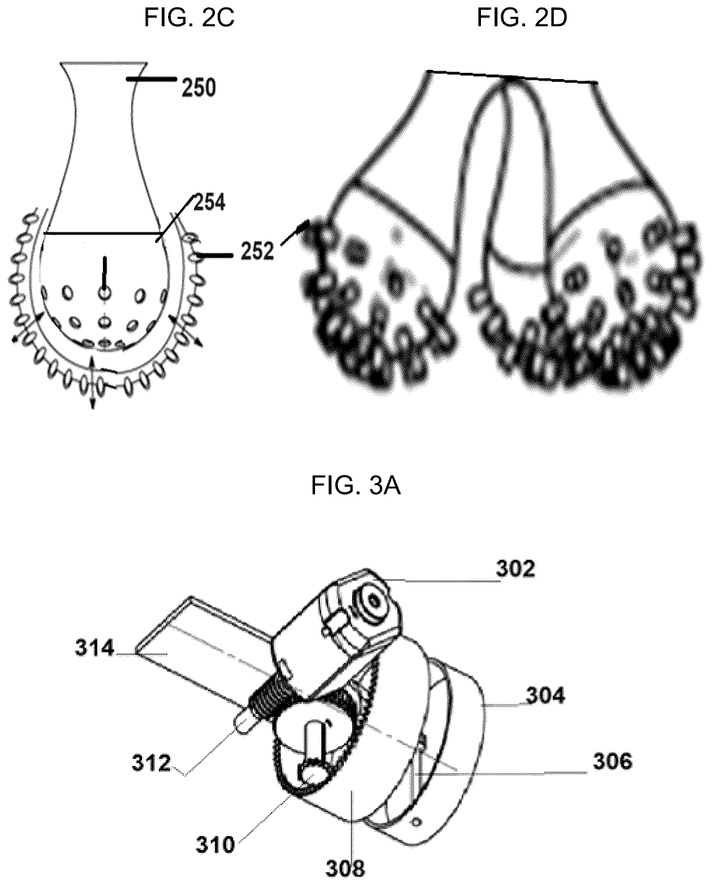

FIG. 2 A is a bottom plan view of the rejuvenation device. FIG. 2, is elaborately explained to comprise ‘202’ as the housing of the skin or facial rejuvenation device, ‘204’ representing sequentially arranged LED lights underneath and underneath the rim of the facial rejuvenation device, ‘206’ is the circular external housing extending to individual finger, and ‘210’ represents the head of an individual rejuvenation finger. In this embodiment, the housing has the central point of the outer wall of the wrench movement rejuvenation unit provided with a phototherapy assembly, with the LEDs arranged uniformly and LED lamp pearls of the housing and the rim located to the cover. The LED lamp beads are blue, red, or purple LEDs or a combination of any color combination. The red-light LED can generate 640 nm to 650 nm red light, can penetrate the dermis, promotes the proliferation of collagen and elastin, reduce wrinkles, and recover the young vitality of the skin; blue light generated by the blue LED can be used for smoothing pox and whelk, tightening pores, acne, closing mouth, flushing and the like, and helps to clean bacteria. ‘208’ is the plurality of individual finger heads with silicon protrusions ‘210’. The silicon protrusions thus create a suction for a minimal period activating the localized blood flow to the skin and helping rejuvenation.

FIG. 2B is an exploded front view of the hand-held massaging device and in detail described as 220′ being the handle to hold the device, ‘222’ being the housing, ‘224’ is the motor rotating the fingers, ‘240’ being the worm wheel gear fixed to the motor and the rejuvenation or massage fingers for horizontal rotation, ‘238’ is the piston fixed between the worm wheel gear and all of the fingers. ‘226’ is the piston head, ‘228’ is the hollow cylindrical silicon rim attached to the rotator and fixed to the piston.'236′ is the common external housing of three fingers, ‘234’ represents the three rejuvenation fingers, and ‘230’ is one of the tiny protrusions placed on the head of each finger.

FIG. 2C and FIG. 2D are the magnified views of one of the three rejuvenation fingers, and the multiple fingers with silicon protrusions respectively. Further in detail explained comprising ‘250’ representing branches of the finger, ‘252 tiny protrusions fixed independently on each finger for vertical rotation in the form of a pull and push mechanism creating a localized suction. The 254 represents the removable sieved cap with a screw locking mechanism with each finger, after removing each cap the silicon protrusions can be cleaned and the cap can be placed back to resume mechanical function.

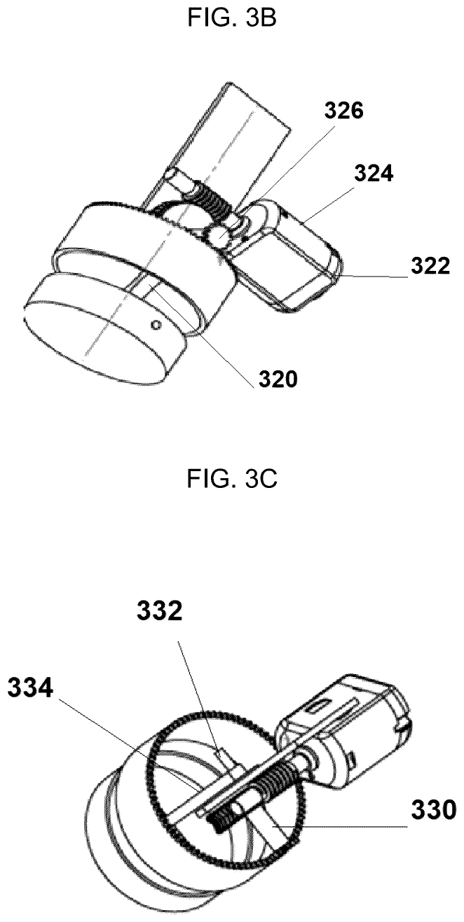

FIG. 3 A is an isometric diagonal left-side view of the push mechanism assembly fixed inside the rejuvenation housing. Further, FIG. 3A is in detail consisting of ‘302’ as the DC-2, ‘304’ as the hollow cylindrical silicon rim, ‘308’ as the rotator, ‘310’ as the protrusion fixed to the worm gear wheel, ‘312’ as the worm gear, and ‘314’ as the part that is holding the gear and fixed to mold for support.

FIG. 3 B is an isometric diagonal right-side view of the push mechanism assembly fixed inside the rejuvenation housing. Further, FIG. 3B is in detail consisting of ‘320’ represented as the piston link, ‘322’ is the DC-2, ‘324’ is the DC-1, and ‘326’ is the protrusion fixed to the worm gear wheel respectively.

FIG. 3C is a trimetric projection of the push mechanism assembly fixed inside the rejuvenation housing. Further, FIG. 3C is in detail consisting of ‘330’ as protrusion of worm gear wheel, ‘332’ is the piston pin head and ‘334’ is the piston link respectively.

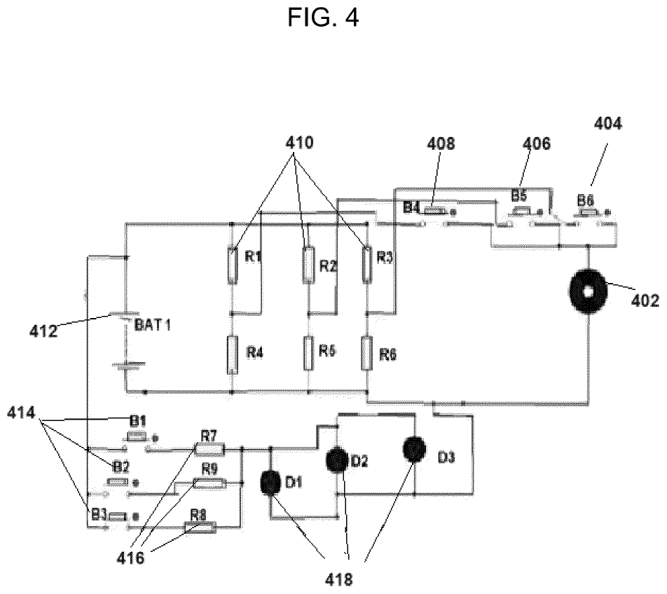

FIG. 4 is related to the circuit diagram concerning the working of the skin rejuvenation. The circuit comprises 9 resistors, 6 buttons, and 3 LEDs. Further FIG. 4 in detail is explained comprising ‘410’ representing resistors R1, R2, and R3 respectively. Further resistors R1, R2, and R3 are connected to battery ‘412’ on the left side and buttons ‘408 B4’, ‘406 B5’, and ‘404 B6’ respectively. For configuring high speed, medium speed, and low speed based on the user's choice, the buttons ‘B4’, ‘B5’, and ‘B5’ can be selected respectively. The circuit also has another set of resistors ‘416’ representing ‘R7’, ‘R8’, and ‘R9’ respectively, connected to ‘414’ representing buttons ‘B1’, ‘B2’, and ‘B3’ respectively, wherein ‘B1’ represents high luminance, ‘B2’ represents medium luminance and ‘B3’ represents low luminance connected to LED's red, purple and blue. ‘418’ represents ‘D1’, ‘D2’ and ‘D3’ respectively representing LEDs. All the components are connected to the ‘402’ representing the DC motor for facilitating horizontal and vertical push and pull pulse movement. The resistors comprise various intensities based on the requirement of the flow of microcurrent. The LED mainly has options like red, purple, and blue representing high, medium, and low frequencies respectively, and LED luminance can be adjusted by three buttons. All the other components are connected to a DC motor and powered by a 12-volt battery. Further, according to the user's requirement, the motor speed can be adjusted by three buttons.

According to some embodiments of the utility model, be provided with on the casing light emitting component with adjust luminance drive module, adjust luminance drive module with the light emitting component electricity is connected, a light emitting component can court the outside emission light of casing.

### Example 1

One of the embodiments, of the present invention relates to the process of working the skin rejuvenation device with the following steps below as EXAMPLE 1, comprising:
i. Step1 is initiated by switching on the rejuvenation device by the user, The elastic rejuvenation needles positioned among the rejuvenation heads first press the skin to enable the skin of the user to have a certain tension degree, and then the rejuvenation heads uniformly rejuvenation the skin of the user through the driving of the rotary driving mechanism for improved blood circulation and detoxification of the skin, resulting in a supple and healthy complexion, and latter increases blood circulation and lymph flow, which prevent wrinkles on the skin.
ii. Step 2, followed by the micro-current feature, helps to boost the production of natural elastin, which further leads to improved elasticity. It also increases collagen synthesis and diminishes wrinkles and fine lines. Thus, it reduces skin roughness and pigmentation,
iii. Step 3 is facilitating the LED light function, based on the functional requirements such as
   a. Red LED helps to stimulate collagen, which is the primary building block of one's skin,
   b. The Blue LED does kill acne-causing bacteria and also helps to improve one's skin texture and tone, and;
   c. The purple LED is for uniform skin tone and reduces mitigation, hyper-pigmentation, and the appearance of age spots.
iv. Step 4 comprises applications of massaging fingers and micro-massaging head are two exquisite features of the device that work wonders. the. Applying both features gives, the skin a flushed, natural glow as all the components are each device component is hypoallergenic and safe to use, ensuring a worry-free experience for every user.

One of the embodiments of the present invention relates to the components of the skin rejuvenation device comprising:
i. an upper base housing,
ii. a motor, and;
iii. gears.

### Example 2

Further, the components are connected, from a mechanical perspective EXAMPLE 2 comprises the following steps:
i. Step 1, comprises an upper base housing, that supports the internal connection between gears and to the upper shell simultaneously.
ii. In step 2, the motor is connected to the worm gear connected to the worm wheel gear and rotates the rotor gear, connected to the rotor holding all three finger housings. Thus, rotating all the fingers horizontally.
iii. Step 3, Additionally, the horizontal rotating gear facilitates the vertical rotation by a push mechanism. The push mechanism further comprises pushing hollow cylindrical silicon rim by piston link and piston head connected in between the worm wheel gear and rotator. The piston head is used for applying pressure on silicon through the piston link. Furthermore, the worm wheel gear transfers electrical power from the motor to the rotator through the piston assembly i.e., the main gear rotates the piston link. It converts the rotatory motion to the linear motion of the piston head, thus, facilitating the vertical motion of the silicon push part.

In the description of the present invention, it should be noted that, for the terms of orientation, such as “central”, “lateral (X)”, “longitudinal (Y)”, “vertical (Z)”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, etc., indicate that the orientation and positional relationship are based on the orientation or positional relationship shown in the drawings, and are only for the convenience of describing the present invention and simplifying the description, but do not indicate or imply that the referred device or element must have a specific orientation, be constructed and operated in a specific orientation, and should not be construed as limiting the specific scope of the present invention.

Furthermore, if the terms “first” and “second” are used for descriptive purposes only, they are not to be construed as indicating or implying relative importance or implicitly indicating the number of technical features. Thus, a definition of “a first” or “a second” feature may explicitly or implicitly include one or more of the features, and in the description of the invention, “a number” means two or more unless explicitly defined otherwise.

In the present invention, unless otherwise expressly specified or limited, the terms “assembled”, “connected”, and “connected” are to be construed broadly, e.g., as meaning fixedly connected, detachably connected, or integrally connected; or maybe a mechanical connection; the two elements can be directly connected or connected through an intermediate medium, and the two elements can be communicated with each other. The specific meanings of the above terms in the present invention can be understood by those of ordinary skill in the art according to specific situations.

1. A hand-held skin rejuvenation device comprising suctional pulse generation.

2. A handheld skin rejuvenation comprising:
v. a housing,
vi. a massage head,
vii. a plurality of fingers,
viii. a detachable mesh cap on each finger,
ix. a plurality of silicon protrusions,
x. a motor,
xi. a plurality of batteries,
xii. a transmission circuit, and;
xiii. light emitting diode.

3. A skin rejuvenation device according to claim 2, provides a uniform rejuvenation, comprising:
i. a housing for holding,
ii. a massage head,
iii. a plurality of fingers connected to housing,
iv. a detachable, mesh cap on each finger for cleaning,
v. a plurality of silicon protrusions for creating suction,
vi. a motor,
vii. a plurality of batteries,
viii. a transmission circuit for facilitating microcurrent,
ix. a worm gearwheel,
x. a piston fixed between the worm wheel gear and fingers,
xi. a piston link,
xii. a rotator,
xiii. a silicon rim attached to the rotator, and;
xiv. a plurality of resistors.

4. A hand-held skin rejuvenation device, according to claim 1, comprising horizontal rotation, with simultaneous vertical push-pull suctional pulse generation.

5. A skin rejuvenation device according to claim 1, comprising vertical push-pull suctional pulse generation created by silicon protrusions.

6. A skin rejuvenation device according to claim 1, comprising vertical push-pull suctional pulse generation created by silicon protrusions with simultaneous horizontal rotation of fingers.

7. A skin rejuvenation device according to claim 2, comprising a transmission of a circuit comprising:
i. a plurality of resistors,
ii. a plurality of buttons, and;
iii. a plurality of light-emitting diodes.

8. A skin rejuvenation hand-held device according to claim 2, comprising of a rotator mechanism, comprising of rotatably disposed housing, a massage head connected to a rotating shaft, a transmission circuit, paired between the silicon protrusions on fingers, and a rotating shaft.

9. A skin rejuvenation device according to claim 2, with speed settings comprising:
i. highspeed,
ii. medium speed,
iii. low speed, and;
iv. a plurality of pulse generation at regular intervals in combination with one of the above three speeds.

10. A rejuvenation fingers according to claim 2, comprising a rotation shaft that can be electrically conductive, and an electrotherapy driving module electrically connected to the rotation shaft is provided in the housing, electrically conductive fingers with silicon protrusions conduct microcurrent pulse generation during rotation.

11. A rejuvenation device according to claim 2, comprising a detachable mesh of individual fingers to facilitate cleaning.

12. A rejuvenation device according to claim 2, in a combination of a light-emitting module with a plurality of colors relating to the intensity comprising:
i. a red color with high-intensity rejuvenation,
ii. a blue color with medium intensity rejuvenation, and;
iii. a violet color with low-intensity rejuvenation.
